(19) United States
(12) Patent Application Publication
(10) Pub. No.: US 2021/0153761 A1
(43) Pub. Date: May 27, 2021

(54) METHOD, SYSTEM AND NON-TRANSITORY COMPUTER-READABLE RECORDING MEDIUM FOR ESTIMATING ARRHYTHMIA BY USING ARTIFICIAL NEURAL NETWORK

(71) Applicant: HUINNO CO., LTD, Seoul (KR)

(72) Inventors: Sung Hoon JUNG, Busan (KR); Jin Kook KIM, Seoul (KR); Yeong Joon GIL, Busan (KR)

(21) Appl. No.: 16/816,581

(22) Filed: Mar. 12, 2020

(30) Foreign Application Priority Data
Nov. 25, 2019 (KR) 10-2019-0152676

(51) Int. Cl. A61B 5/04; A61B 5/0402; A61B 5/0245; A61B 5/00; G06N 3/08; G06N 3/04; G06N 20/20

(52) U.S. Cl. CPC A61B 5/04012; A61B 5/0402; A61B 5/0245; G06N 20/20; G06N 3/08; G06N 3/0454; A61B 5/7267

(57) ABSTRACT

A method for estimating arrhythmia comprises analyzing a target biosignal of a subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal corresponds to the first type of arrhythmic state, and a second score for whether at least a part of the target biosignal corresponds to the second type of arrhythmic state, respectively; and estimating types of arrhythmic state to which at least a part of the target biosignal corresponds, on the basis of the scores and a training index of each of a plurality of artificial neural networks including the first and second artificial neural networks.

### CROSS-REFERENCE TO RELATED APPLICATIONS

This application claims priority to Korean Patent Application No. 10-2019-0152676 filed on Nov. 25, 2019, the entire contents of which are hereby incorporated by reference.

### TECHNICAL FIELD

The present invention relates to a method, system, and non-transitory computer-readable recording medium for estimating arrhythmia using artificial neural networks.

### RELATED ART

Due to the recent rapid development of science and technology, the quality of life of the entire human race is improving, and many changes have occurred in the medical environment. In the past, image reading was possible after a few hours or days from when medical images such as X-rays, CTs, and fMRIs were taken in a hospital.

Recently, as wearable devices that form contacts with various body parts (e.g., chest, wrist, ankle, etc.) of a subject to measure biosignals (e.g., ECG signals) have become widespread, the techniques for constantly measuring or analyzing biosignals in daily life have been introduced. In particular, the techniques for recognizing arrhythmia by analyzing constantly measured electrocardiogram (ECG) signals have attracted attention.

Conventionally, skilled medical attendants rely on the traditional method of discriminating arrhythmia by personally reading ECG signals on the basis of their clinical judgment. However, in recent years, the techniques for determining the presence or absence of arrhythmia or recognizing the type of arrhythmia by analyzing ECG signals using rapidly evolving artificial intelligence (or artificial neural network) technology have been introduced.

Specifically, arrhythmia can be subdivided into ten or more different types according to its characteristics. In order to accurately recognize the type of arrhythmic state to which an ECG signal correspond, there is a need to train artificial neural networks using a wide variety of data regarding ECG signals corresponding to a normal state and ECG signals corresponding to various types of arrhythmic state.

As an example of related conventional techniques, according to a technique disclosed in Korean Patent Laid-Open Publication No. 2019-88680, an apparatus for generating an artificial neural network has been introduced, which comprises: an input unit for receiving a blood pressure signal obtained by measuring a patient's blood pressure N times at every predetermined time interval for a predetermined period of time; a parameter acquisition unit for acquiring a blood pressure parameter from the blood pressure signal; and a generation unit for generating, on the basis of the blood pressure parameter and whether the patient develops a ventricular arrhythmia, an artificial neural network trained on a correlation between the blood pressure parameter and whether the patient has developed the ventricular arrhythmia, wherein the blood pressure parameter includes information on the degree of blood pressure change, indicating the degree of change by which the measured blood pressure signal has changed from a blood pressure signal measured immediately before.

However, according to the techniques introduced so far as well as the above-described conventional technique, the artificial neural network is formed as a single multiple classification-based network that decides both the presence or absence of an arrhythmic state and a plurality of types of the arrhythmic state, so that when the number of classifications is increased after the form of the network has been determined, the sensitivity of each classification is lowered due to the limited classification capacity of the network. In order to maintain the sensitivity of each classification, it is possible to consider increasing the classification capacity of the network (e.g., increasing the number of hidden layers or increasing the number of kernels for feature extraction). However, as the complexity of the network increases, there may arise problems that training is improperly performed or more training data are required.

Further, with the artificial neutral network being formed as a single multiple classification-based network as above, the entire result regarding the arrhythmia may come out poorly when the network is improperly trained due to asymmetry of the training data or the like.

In this connection, the inventor(s) present a novel and inventive technique for accurately estimating the presence or absence of an arrhythmic state and the types of the arrhythmic state, using a plurality of binary classification-based artificial neural networks that are constructed in a parallel manner and respectively trained regarding the presence or absence of the arrhythmic state or the types of the arrhythmic state.

### SUMMARY

One object of the present invention is to solve all the above-described problems.

Another object of the invention is to estimate arrhythmia with high sensitivity using artificial neural networks that are based on binary classification regarding the presence or absence of an arrhythmic state or the types of the arrhythmic state and constructed in a parallel manner, even when the number of classifications regarding the presence or absence of the arrhythmic state or the types of the arrhythmic state is increased.

Yet another object of the invention is to form customized artificial neural networks for estimating arrhythmia, such that artificial neural networks based on binary classification regarding the presence or absence of an arrhythmic state or the types of the arrhythmic state are constructed in a parallel manner and may be added or removed depending on the purpose of use, the purpose of examination, or the like.

The representative configurations of the invention to achieve the above objects are described below.

According to one aspect of the invention, there is provided a method for estimating arrhythmia using artificial neural networks, comprising the steps of: analyzing a target biosignal of a subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal corresponds to the first type of arrhythmic state, and a second score for whether at least a part of the target biosignal corresponds to the second type of arrhythmic state, respectively; and estimating types of arrhythmic state to which at least a part of the target biosignal corresponds, on the basis of the scores and a training index of each of a plurality of artificial neural networks including the first and second artificial neural networks.

According to another aspect of the invention, there is provided a method for estimating arrhythmia using artificial neural networks, comprising the steps of: analyzing a target biosignal of a subject using a first artificial neural network based on binary classification and trained on data regarding biosignals corresponding to a specific type of arrhythmic state, and a second artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively; and estimating types of arrhythmic state to which at least a part of the target biosignal corresponds, on the basis of the scores and a training index of each of a plurality of artificial neural networks including the first and second artificial neural networks.

According to yet another aspect of the invention, there is provided a system for estimating arrhythmia using artificial neural networks, comprising: a biosignal acquisition unit configured to acquire a target biosignal measured from a subject; a score calculation unit configured to analyze the target biosignal of the subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal of the subject corresponds to the first type of arrhythmic state, and a second score for whether at least a part of the target biosignal of the subject corresponds to the second type of arrhythmic state, respectively; and a state estimation unit configured to estimate types of arrhythmic state to which at least a part of the target biosignal corresponds, on the basis of the scores and a training index of each of a plurality of artificial neural networks including the first and second artificial neural networks.

According to still another aspect of the invention, there is provided a system for estimating arrhythmia using artificial neural networks, comprising: a biosignal acquisition unit configured to acquire a target biosignal measured from a subject; a score calculation unit configured to analyze the target biosignal using a first artificial neural network based on binary classification and trained on data regarding biosignals corresponding to a specific type of arrhythmic state, and a second artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively; and a state estimation unit configured to estimate types of arrhythmic state to which at least a part of the target biosignal corresponds, on the basis of the scores and a training index of each of a plurality of artificial neural networks including the first and second artificial neural networks.

In addition, there are further provided other methods and systems to implement the invention, as well as non-transitory computer-readable recording media having stored thereon computer programs for executing the methods.

According to the invention, it is possible to estimate arrhythmia with high sensitivity using artificial neural networks that are based on binary classification regarding the presence or absence of an arrhythmic state or the types of the arrhythmic state and constructed in a parallel manner, even when the number of classifications regarding the presence or absence of the arrhythmic state or the types of the arrhythmic state is increased.

According to the invention, it is possible to form customized artificial neural networks for estimating arrhythmia, such that artificial neural networks based on binary classification regarding the presence or absence of an arrhythmic state or the types of the arrhythmic state are constructed in a parallel manner and may be added or removed depending on the purpose of use, the purpose of examination, or the like.

### BRIEF DESCRIPTION OF THE DRAWINGS

FIG. 1 schematically shows the configuration of an entire system according to the invention;

FIG. 2 illustratively shows the internal configuration of an arrhythmia estimation system according to one embodiment of the invention;

FIGS. 3A and 3B illustratively show the configurations of artificial neural networks used to estimate arrhythmia according to one embodiment of the invention;

FIGS. 4 and 5 illustratively show how to estimate types of arrhythmia using a plurality of artificial neural networks according to one embodiment of the invention; and

FIG. 6 shows test results in which sensitivity of an artificial neural network is decreased as the number of classifications thereof is increased.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each of the disclosed embodiments may also be modified without departing from the spirit and scope of the invention. Therefore, the following detailed description is not to be taken in a limiting sense, and the scope of the invention, if properly described, is limited only by the appended claims together with all equivalents thereof. In the drawings, like reference numerals refer to the same or similar functions throughout the several views.

Hereinafter, preferred embodiments of the present invention will be described in detail with reference to the accompanying drawings to enable those skilled in the art to easily implement the invention.

### Configuration of the Entire System

Preferred embodiments of an arrhythmia estimation system according to the invention will be discussed in detail below.

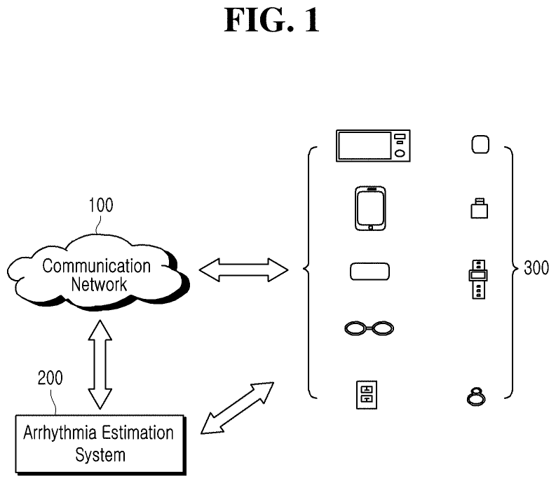

FIG. 1 schematically shows the configuration of the entire system according to the invention.

As shown in FIG. 1, the entire system according to one embodiment of the invention may comprise a communication network 100, an arrhythmia estimation system 200, and a device 300.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may include a known wireless local area network such as Wi-Fi, Wi-Fi Direct, LTE Direct, and Bluetooth. However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Next, the arrhythmia estimation system 200 according to one embodiment of the invention may function to analyze a target biosignal of a subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal corresponds to the first type of arrhythmic state, and a second score for whether at least a part of the target biosignal corresponds to the second type of arrhythmic state, respectively, and to estimate types of arrhythmic state to which at least a part of the target biosignal corresponds, on the basis of the scores and a training index of each of a plurality of artificial neural networks including the first and second artificial neural networks.

Next, the arrhythmia estimation system 200 according to one embodiment of the invention may function to analyze a target biosignal of a subject using a third artificial neural network based on binary classification and trained on data regarding biosignals corresponding to a specific type of arrhythmic state, and a fourth artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively, and to estimate types of arrhythmic state to which at least a part of the target biosignal corresponds, on the basis of the scores and a training index of each of a plurality of artificial neural networks including the third and fourth artificial neural networks.

The binary classification-based artificial neural network according to one embodiment of the invention may mean an artificial neural network that outputs, when data regarding a certain biosignal are inputted, a result regarding to which one of two classifications the biosignal belongs. For example, when data regarding a certain biosignal are inputted to a binary classification-based artificial neural network having two classifications of normality and abnormality, and the value outputted as a result is 0.7, the result may mean normality with a chance of 70%.

The functions of the arrhythmia estimation system 200 according to the invention will be discussed in more detail below. Meanwhile, although the arrhythmia estimation system 200 has been described as above, the above description is illustrative and it will be apparent to those skilled in the art that at least a part of the functions or components required for the arrhythmia estimation system 200 may be implemented or included in the device 300, as necessary.

Next, the device 300 according to one embodiment of the invention is digital equipment that may function to connect to and then communicate with the arrhythmia estimation system 200, and any type of digital equipment having a memory means and a microprocessor for computing capabilities may be adopted as the device 300 according to the invention. The device 300 may be a wearable device such as smart glasses, a smart watch, a smart band, a smart ring, and a smart necklace, or may be a somewhat traditional device such as a smart phone, a smart pad, a desktop computer, a notebook computer, a workstation, a personal digital assistant (PDA), a web pad, and a mobile phone.

Particularly, the device 300 according to one embodiment of the invention may include a sensing means (e.g., a contact electrode, an imaging device, etc.) for acquiring a biosignal from a human body, and may include a display means for providing a user with a variety of information on biosignal measurements.

Further, according to one embodiment of the invention, the device 300 may include an application for performing the functions according to the invention. The application may reside in the device 300 in the form of a program module. The characteristics of the program module may be generally similar to those of a biosignal acquisition unit 210, a score calculation unit 220, a state estimation unit 230, a communication unit 240, and a control unit 250 of the arrhythmia estimation system 200 to be described below. Here, at least a part of the application may be replaced with a hardware device or a firmware device that may perform a substantially equal or equivalent function, as necessary.

### Configuration of the Arrhythmia Estimation System

Hereinafter, the internal configuration of the arrhythmia estimation system 200 crucial for implementing the invention and the functions of the respective components thereof will be discussed.

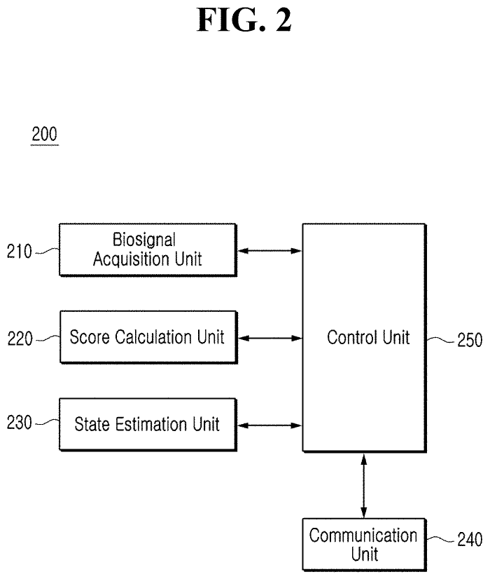

FIG. 2 illustratively shows the internal configuration of the arrhythmia estimation system according to one embodiment of the invention.

Referring to FIG. 2, the arrhythmia estimation system 200 according to one embodiment of the invention may comprise a biosignal acquisition unit 210, a score calculation unit 220, a state estimation unit 230, a communication unit 240, and a control unit 250. According to one embodiment of the invention, at least some of the biosignal acquisition unit 210, the score calculation unit 220, the state estimation unit 230, the communication unit 240, and the control unit 250 may be program modules to communicate with an external system (not shown). The program modules may be included in the arrhythmia estimation system 200 in the form of operating systems, application program modules, and other program modules, while they may be physically stored in a variety of commonly known storage devices. Further, the program modules may also be stored in a remote storage device that may communicate with the arrhythmia estimation system 200. Meanwhile, such program modules may include, but not limited to, routines, subroutines, programs, objects, components, data structures and the like for performing specific tasks or executing specific abstract data types as will be described below in accordance with the invention.

First, the biosignal acquisition unit 210 according to one embodiment of the invention may function to acquire a biosignal from the device 300 or at least one measurement module (not shown) (e.g., a biosignal measurement sensor module) that is in contact with a body part of a subject. The biosignal according to one embodiment of the invention may include a signal regarding at least one of an electrocardiogram (ECG), an electromyogram (EMG), an electroencephalogram (EEG), a photoplethysmogram (PPG), a heartbeat, a body temperature, a blood sugar level, a pupil change, a blood pressure level, and a blood oxygen content.

For example, the biosignal acquisition unit 210 according to one embodiment of the invention may acquire an ECG signal of the subject as the above biosignal from at least one measurement module that is connected via a wireless communication network (e.g., a known wireless local area network such as Wi-Fi, Wi-Fi Direct, LTE Direct, and Bluetooth).

Further, the biosignal acquisition unit 210 according to one embodiment of the invention may acquire the biosignal of the subject from at least one recording device (e.g., a server, cloud, etc.) in which the biosignal of the subject is pre-stored.

Next, the score calculation unit 220 according to one embodiment of the invention may function to analyze a target biosignal of the subject acquired by the biosignal acquisition unit 210, using a first artificial neural network based on binary classification and trained on data regarding biosignals (e.g., ECG signals) corresponding to a first type of arrhythmic state, and a second artificial neural network based on binary classification and trained on data regarding biosignals corresponding to a second type of arrhythmic state, thereby calculating a first score for whether at least a part of the target biosignal corresponds to the first type of arrhythmic state, and a second score for whether at least a part of the target biosignal corresponds to the second type of arrhythmic state, respectively. The scores according to one embodiment of the invention may encompass a value regarding at least one of a probability, a vector, a matrix, and a coordinate regarding correspondence (or non-correspondence) to a specific type of arrhythmic state.

For example, the score calculation unit 220 according to one embodiment of the invention may implement the first and second binary classification-based artificial neural networks on the basis of an input layer, at least one hidden layer, and an output layer, and may train the first and second artificial neural networks on data regarding ECG signals (i.e., biosignals) corresponding to the first type of arrhythmic state and data regarding ECG signals corresponding to the second type of arrhythmic state, respectively.

Next, the score calculation unit 220 according to one embodiment of the invention may calculate a probability that is outputted when at least a part of the target ECG signal is inputted to the first binary classification-based artificial neural network trained on the data regarding the ECG signals corresponding to the first type of arrhythmic state (e.g., a probability of correspondence to the first type of arrhythmic state) as the first score, and may calculate a probability that is outputted when at least a part of the target ECG signal is inputted to the second binary classification-based artificial neural network trained on the data regarding the ECG signals corresponding to the second type of arrhythmic state (e.g., a probability of correspondence to the second type of arrhythmic state) as the second score.

Further, the score calculation unit 220 according to one embodiment of the invention may analyze a target biosignal of the subject using a third artificial neural network based on binary classification and trained on data regarding biosignals corresponding to a specific type of arrhythmic state, and a fourth artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively.

For example, the score calculation unit 220 according to one embodiment of the invention may implement the third and fourth binary classification-based artificial neural networks on the basis of an input layer, at least one hidden layer, and an output layer, and may train the third and fourth artificial neural networks on data regarding ECG signals corresponding to the specific type of arrhythmic state and at least one of data regarding normal state ECG signals and data regarding arrhythmic state ECG signals, respectively.

Next, the score calculation unit 220 according to one embodiment of the invention may calculate a probability that is outputted when at least a part of the target ECG signal of the subject is inputted to the third binary classification-based artificial neural network trained on the data regarding the ECG signals corresponding to the specific type of arrhythmic state (e.g., a probability of correspondence to the specific type of arrhythmic state) as a third score, and may calculate a probability that is outputted when at least a part of the target ECG signal is inputted to the fourth binary classification-based artificial neural network trained on at least one of the data regarding the normal state ECG signals and the data regarding the arrhythmic state ECG signals (e.g., a probability of correspondence to a normal state or an arrhythmic state) as a fourth score.

However, it is noted that the techniques for implementing and training the first to fourth artificial neural networks according to the invention are not necessarily limited to the foregoing, and may be changed to convolutional neural networks (CNNs), recurrent neural networks (RNNs), auto-encoders, and the like without limitation, as long as the objects of the invention may be achieved.

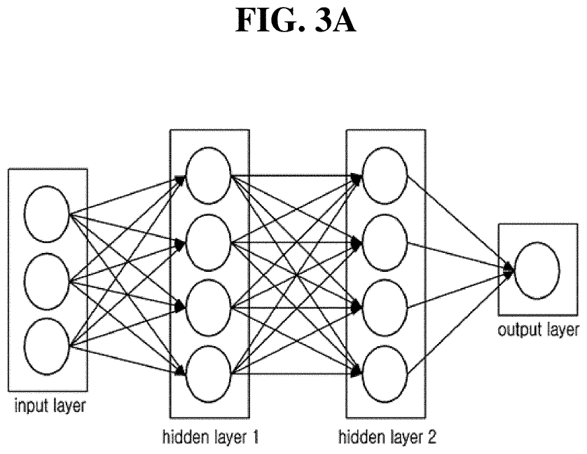

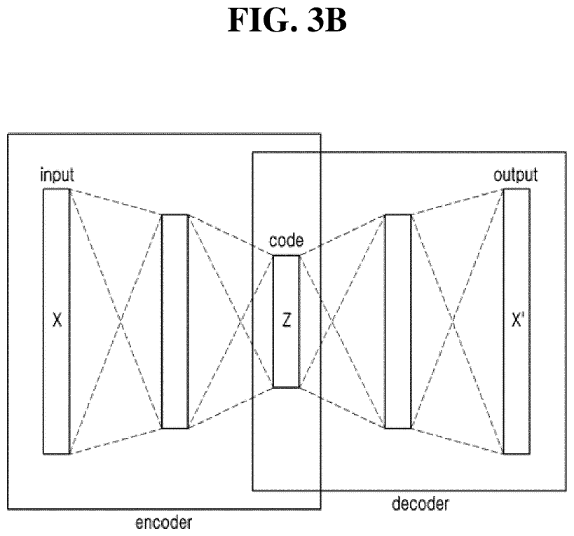

FIGS. 3A and 3B illustratively show the configurations of artificial neural networks used to estimate arrhythmia according to one embodiment of the invention.

First, referring to FIG. 3A, an artificial neural network used to discriminate whether arrhythmia is present may be implemented such that an input layer, at least one hidden layer, and an output layer are sequentially combined. The dimensions of the input layer, hidden layer, and output layer constituting the artificial neutral network may be equal or different.

Referring further to FIG. 3A, the score calculation unit 220 according to one embodiment of the invention may train the above artificial neural network on normal state biosignals and arrhythmic state biosignals. For example, the artificial neural network may be trained by labeling a normal state biosignal as 0 and an arrhythmic state biosignal as 1.

Accordingly, referring to FIG. 3A, when a target biosignal of a subject is inputted to the above artificial neural network, the score calculation unit 220 according to one embodiment of the invention may cause the artificial neural network to output a probability that the target biosignal corresponds to a normal state biosignal or an arrhythmic state biosignal. For example, the target biosignal is likely to be a normal state biosignal when the outputted probability is close to 0 in relation to 0.5, and the target biosignal is likely to be an arrhythmic state biosignal when the outputted probability is close to 1 in relation to 0.5.

Next, referring to FIG. 3B, an artificial neural network used to discriminate whether arrhythmia is present may be implemented such that an encoder and a decoder are sequentially combined and the dimensions of an input end of the encoder and an output end of the decoder are equal. The artificial neural network may be trained such that a biosignal X inputted to the encoder becomes identical to a biosignal X′ outputted from the decoder.

Referring further to FIG. 3B, the score calculation unit 220 according to one embodiment of the invention may train the above artificial neural network only on normal state biosignals. Assuming that the artificial neural network has been successfully trained according to the above training method, the difference between biosignals inputted to and outputted from the artificial neural network may be relatively small when the inputted biosignal corresponds to a normal state, and may be relatively large when the inputted biosignal corresponds to an arrhythmic state.

Accordingly, referring to FIG. 3B, when a target biosignal of a subject is inputted to the above artificial neural network, the score calculation unit 220 according to one embodiment of the invention may determine that the target biosignal is a normal state biosignal when the difference between the inputted target biosignal and a biosignal outputted from the artificial neural network is less than a predetermined level, and may determine that the target biosignal is an arrhythmic state biosignal when the difference is not less than the predetermined level. Further, when a target biosignal of the subject is inputted to the above artificial neural network, the score calculation unit 220 according to one embodiment of the invention may cause the artificial neural network to output a probability that the target biosignal corresponds to a normal state biosignal or an arrhythmic state biosignal, on the basis of the difference between the inputted target biosignal and a biosignal outputted from the artificial neural network.

Meanwhile, the score calculation unit 220 according to one embodiment of the invention may analyze a target biosignal of a subject using an artificial neural network based on binary classification and trained on at least one of data regarding normal state biosignals and data regarding arrhythmic state biosignals, thereby predetermining whether at least a part of the target biosignal of the subject corresponds to an arrhythmic state, and may analyze the biosignal determined to correspond to the arrhythmic state using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the biosignal determined to correspond to the arrhythmic state corresponds to the first type of arrhythmic state, and a second score for whether at least a part of the biosignal determined to correspond to the arrhythmic state corresponds to the second type of arrhythmic state, respectively. That is, it is possible to obtain a result efficiently and quickly by performing the above-described score calculation on a biosignal that is predetermined to correspond to an arrhythmic state.

Next, the state estimation unit 230 according to one embodiment of the invention may function to estimate types of arrhythmic state to which at least a part of the target biosignal corresponds, on the basis of the scores and a training index of each of a plurality of artificial neural networks including the first and second artificial neural networks or the third and fourth artificial neural networks. The training index according to one embodiment of the invention may encompass at least one of precision, recall, and accuracy of an artificial neural network.

For example, the state estimation unit 230 according to one embodiment of the invention may estimate to which one of a plurality of types of arrhythmic state at least a part of the target biosignal of the subject corresponds, on the basis of values calculated from the scores and the training index of each of the plurality of artificial neural networks.

Further, the state estimation unit 230 according to one embodiment of the invention may determine, as information to be provided, information on at least one type of arrhythmic state that satisfies predetermined criteria, among the estimated types of arrhythmic state to which at least a part of the target biosignal corresponds.

For example, the state estimation unit 230 according to one embodiment of the invention may determine, as the information to be provided, information on rankings and names of types of arrhythmic state that correspond to the calculated values not less than 0.5, among the estimated types of arrhythmic state. That is, the state estimation unit 230 according to one embodiment of the invention may determine that types of arrhythmic state corresponding to the calculated values less than 0.5 cannot be accurately estimated using the artificial neural networks, and exclude them from the information to be provided.

Next, the communication unit 240 according to one embodiment of the invention may function to enable data transmission/reception from/to the biosignal acquisition unit 210, the score calculation unit 220, and the state estimation unit 230.

Lastly, the control unit 250 according to one embodiment of the invention may function to control data flow among the biosignal acquisition unit 210, the score calculation unit 220, the state estimation unit 230, and the communication unit 240. That is, the control unit 250 according to the invention may control data flow into/out of the arrhythmia estimation system 200 or data flow among the respective components of the arrhythmia estimation system 200, such that the biosignal acquisition unit 210, the score calculation unit 220, the state estimation unit 230, and the communication unit 240 may carry out their particular functions, respectively.

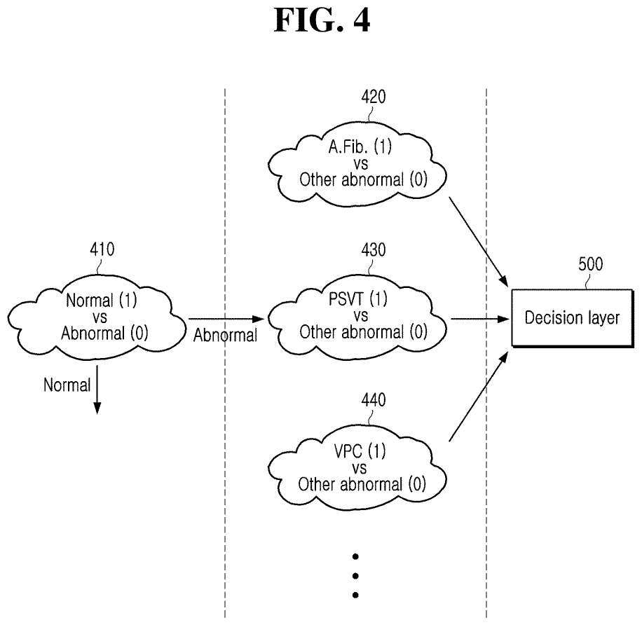

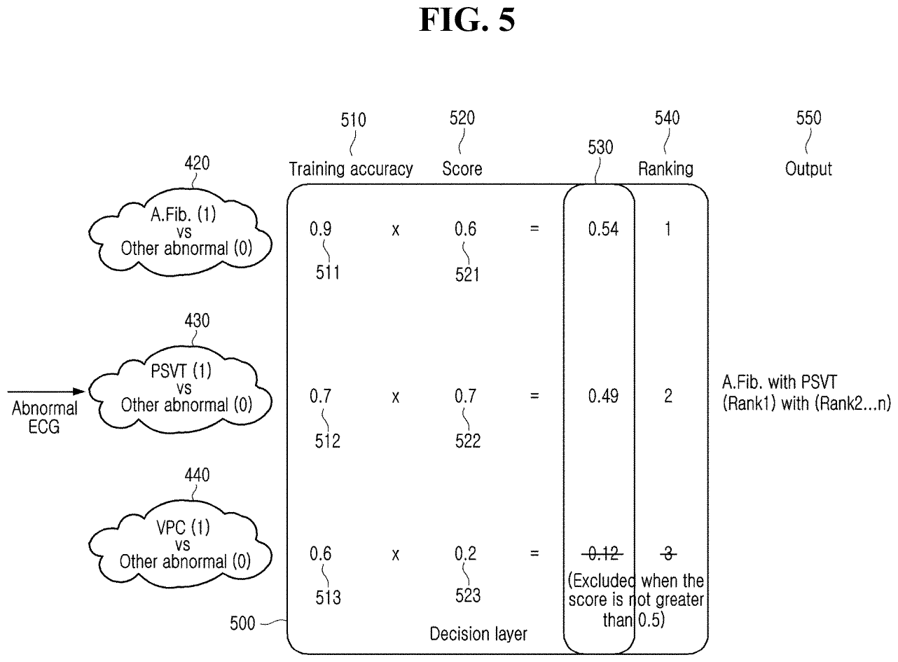

FIGS. 4 and 5 illustratively show how to estimate an arrhythmic state according to one embodiment of the invention.

First, referring to FIGS. 4 and 5, according to one embodiment of the invention, a target ECG signal measured from a subject may be acquired as a biosignal.

Next, according to one embodiment of the invention, the acquired target ECG signal of the subject may be analyzed using an artificial neural network 410 based on binary classification and trained on at least one of data regarding normal state ECG signals and data regarding arrhythmic state ECG signals, thereby predetermining whether at least a part of the target ECG signal corresponds to the arrhythmic state. That is, the target ECG signal at least a part of which corresponds to an arrhythmic state may be provided as an input for each of first to third artificial neural networks to be described below.

Next, according to one embodiment of the invention, the predetermined ECG signal corresponding to the arrhythmic state may be analyzed using a first artificial neural network 420 based on binary classification and trained on data regarding ECG signals corresponding to atrial fibrillation (AFib) (i.e., a first type of arrhythmic state), a second artificial neural network 430 based on binary classification and trained on data regarding ECG signals corresponding to paroxysmal supra ventricular tachycardia (PSVT) (i.e., a second type of arrhythmic state), and a third artificial neural network 440 based on binary classification and trained on data regarding ECG signals corresponding to ventricular premature complexes (VPCs) (i.e., a third type of arrhythmic state), thereby calculating a first score ((a) of 450) for whether at least a part of the ECG signal corresponding to the arrhythmic state corresponds to AFib (i.e., the first type of arrhythmic state), a second score ((b) of 450) for whether at least a part of the ECG signal corresponding to the arrhythmic state corresponds to PSVT (i.e., the second type of arrhythmic state), and a third score ((c) of 450) for whether at least a part of the ECG signal corresponding to the arrhythmic state corresponds to VPCs (i.e., the third type of arrhythmic state), respectively.

Next, when values 530 calculated from a training accuracy 511 of the first artificial neural network and the first score 521, calculated from a training accuracy 512 of the second artificial neural network and the second score 522, and calculated from a training accuracy 513 of the third artificial neural network and the third score 523 are obtained as 0.54, 0.49, and 0.12, respectively, a ranking 540 of each type of arrhythmic state may be determined on the basis of the values 530, and information on the determined rankings 540 and names 550 of the corresponding types of arrhythmic state may be provided. Meanwhile, it may be determined that the types of arrhythmic state for which the calculated values 530 are not greater than a predetermined level (e.g., 0.5) cannot be estimated using the corresponding artificial neural networks, and the determined types of arrhythmic state may be excluded from the provided information.

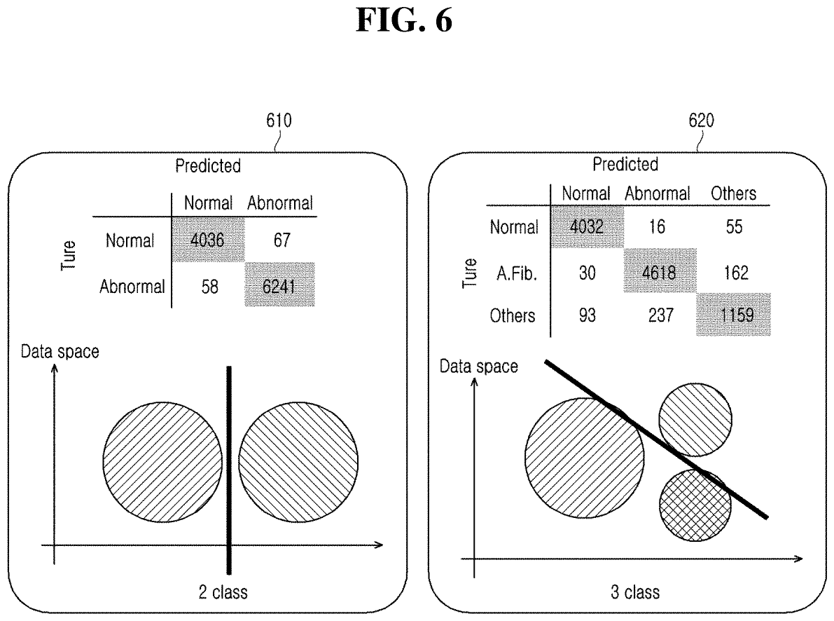

FIG. 6 shows test results in which sensitivity of an artificial neural network is decreased as the number of classifications thereof is increased.

Referring to FIG. 6, a test result 610 is shown in which predetermined ECG data are classified using an artificial neural network based on binary classification regarding a normal state and an arrhythmic state, and a test result 620 is shown in which the predetermined ECG data are classified using an artificial neural network based on multiple classification regarding a normal state, AFib, and other states.

Sensitivity of the normal state in the binary classification-based artificial neural network is calculated as 0.99 (i.e., 6241/(6241+58)) and sensitivity of AFib in the multiple classification-based artificial neural network is calculated as 0.96 (i.e., 4618/(30+4618+162)). Thus, it can be seen that the sensitivity of the multiple classification-based artificial neural network is lower than that of the binary classification-based artificial neural network. According to one embodiment of the invention, binary classification-based artificial neural networks may be constructed in a parallel manner to increase the number of classifications while taking advantage of the higher sensitivity of the binary classification, so that arrhythmia can be estimated more accurately (specifically, with higher sensitivity) than a single multiple classification-based artificial neural network.

Although the embodiments in which arrhythmia is estimated using artificial neural networks have been mainly described above, it is noted that the present invention is not necessarily limited only to arrhythmia but may be utilized for other diseases (e.g., for estimating the presence or absence of a respiratory disease and the type of the disease), other technical fields (e.g., the field of instrument abnormality diagnosis in which at least one of vibration data and sound data acquired from a plurality of sensors are inputted to a plurality of artificial neural networks to estimate the presence or absence of abnormality of an instrument and the type of the abnormality on the basis of results outputted therefrom), and the like without limitation, as long as the objects of the invention may be achieved.

The embodiments according to the invention as described above may be implemented in the form of program instructions that can be executed by various computer components, and may be stored on a non-transitory computer-readable recording medium. The non-transitory computer-readable recording medium may include program instructions, data files, data structures and the like, separately or in combination. The program instructions stored on the non-transitory computer-readable recording medium may be specially designed and configured for the present invention, or may also be known and available to those skilled in the computer software field. Examples of the non-transitory computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be configured to operate as one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for estimating arrhythmia using artificial neural networks, comprising the steps of:
analyzing a target biosignal of a subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal corresponds to the first type of arrhythmic state, and a second score for whether at least a part of the target biosignal corresponds to the second type of arrhythmic state, respectively; and
estimating types of arrhythmic state to which at least a part of the target biosignal corresponds, on the basis of the scores and a training index of each of a plurality of artificial neural networks including the first and second artificial neural networks.

2. The method of claim 1, wherein the calculating step comprises the step of analyzing the target biosignal using an artificial neural network based on binary classification and trained on at least one of data regarding normal state biosignals and data regarding arrhythmic state biosignals, thereby predetermining whether at least a part of the target biosignal corresponds to an arrhythmic state.

3. The method of claim 1, wherein the estimating step comprises the step of providing information on at least one type of arrhythmic state that satisfies predetermined criteria, among the estimated types of arrhythmic state to which the target biosignal corresponds.

4. The method of claim 1, wherein the score includes a probability of correspondence to each of the types of arrhythmic state.

5. The method of claim 1, wherein the training index includes a training accuracy of each of the artificial neural networks.

6. A non-transitory computer-readable recording medium having stored thereon a computer program for executing the method of claim 1.

7. A method for estimating arrhythmia using artificial neural networks, comprising the steps of:
analyzing a target biosignal of a subject using a first artificial neural network based on binary classification and trained on data regarding biosignals corresponding to a specific type of arrhythmic state, and a second artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively; and
estimating types of arrhythmic state to which at least a part of the target biosignal corresponds, on the basis of the scores and a training index of each of a plurality of artificial neural networks including the first and second artificial neural networks.

8. The method of claim 7, wherein the estimating step comprises the step of providing information on at least one type of arrhythmic state that satisfies predetermined criteria, among the estimated types of arrhythmic state to which the target biosignal corresponds.

9. The method of claim 7, wherein the score includes a probability of correspondence to each of the types of arrhythmic state.

10. The method of claim 7, wherein the training index includes a training accuracy of each of the artificial neural networks.

11. A non-transitory computer-readable recording medium having stored thereon a computer program for executing the method of claim 7.

12. A system for estimating arrhythmia using artificial neural networks, comprising:
a biosignal acquisition unit configured to acquire a target biosignal measured from a subject;
a score calculation unit configured to analyze the target biosignal of the subject using a first artificial neural network based on binary classification and trained on data regarding biosignals corresponding to a first type of arrhythmic state, and a second artificial neural network based on binary classification and trained on data regarding biosignals corresponding to a second type of arrhythmic state, thereby calculating a first score for whether at least a part of the target biosignal of the subject corresponds to the first type of arrhythmic state, and a second score for whether at least a part of the target biosignal of the subject corresponds to the second type of arrhythmic state, respectively; and
a state estimation unit configured to estimate types of arrhythmic state to which at least a part of the target biosignal corresponds, on the basis of the scores and a training index of each of a plurality of artificial neural networks including the first and second artificial neural networks.

13. The system of claim 12, wherein the score calculation unit is configured to analyze the target biosignal using an artificial neural network based on binary classification and trained on at least one of data regarding normal state biosignals and data regarding arrhythmic state biosignals, thereby predetermining whether at least a part of the target biosignal corresponds to an arrhythmic state.

14. The system of claim 12, wherein the state estimation unit is configured to provide information on at least one type of arrhythmic state that satisfies predetermined criteria, among the estimated types of arrhythmic state to which the target biosignal corresponds.

15. The system of claim 12, wherein the score includes a probability of correspondence to each of the types of arrhythmic state.

16. The system of claim 12, wherein the training index includes a training accuracy of each of the artificial neural networks.

17. A system for estimating arrhythmia using artificial neural networks, comprising:
a biosignal acquisition unit configured to acquire a target biosignal measured from a subject;
a score calculation unit configured to analyze the target biosignal using a first artificial neural network based on binary classification and trained on data regarding biosignals corresponding to a specific type of arrhythmic state, and a second artificial neural network based on binary classification and trained on at least one of data regarding normal state biosignals and data regarding arrhythmic state biosignals, thereby calculating a score for whether at least a part of the target biosignal corresponds to the specific type of arrhythmic state, and a score for whether at least a part of the target biosignal corresponds to an arrhythmic state, respectively; and
a state estimation unit configured to estimate types of arrhythmic state to which at least a part of the target biosignal corresponds, on the basis of the scores and a training index of each of a plurality of artificial neural networks including the first and second artificial neural networks.

18. The system of claim 17, wherein the state estimation unit is configured to provide information on at least one type of arrhythmic state that satisfies predetermined criteria, among the estimated types of arrhythmic state to which the target biosignal corresponds.

19. The system of claim 17, wherein the score includes a probability of correspondence to each of the types of arrhythmic state.

20. The system of claim 17, wherein the training index includes a training accuracy of each of the artificial neural networks.
